(19) United States
(12) Patent Application Publication
(10) Pub. No.: US 2011/0166115 A1
(43) Pub. Date: Jul. 7, 2011

(54) USE OF MIFEPRISTONE FOR THE TREATMENT OF AMYOTROPHIC LATERAL SCLEROSIS

(75) Inventor: Joseph Belanoff, Woodside, CA (US)

(73) Assignee: Corcept Therapeutics, Inc., Menlo Park, CA (US)

(21) Appl. No.: 13/001,211

(22) PCT Filed: Jun. 30, 2009
(86) PCT No.: PCT/US09/49273

§ 371 Date: Mar. 24, 2011

Related U.S. Application Data
(60) Provisional application No. 61/077,248, filed on Jul. 1, 2008.

(51) Int. Cl. A61K 31/57; A61P 25/28

(52) U.S. Cl. 514/177

(57) ABSTRACT

The invention generally pertains to the discovery that agents capable of inhibiting the binding of cortisol to its receptor can be used in methods for treating patients diagnosed with Amyotrophic Lateral Sclerosis (ALS).

### CROSS-REFERENCES TO RELATED APPLICATIONS

This application claims benefit of U.S. Provisional Application No. 61/077,248 filed on Jul. 1, 2008, the disclosure of which is hereby incorporated by reference in its entirety.

### FIELD OF INVENTION

The invention relates to the discovery that an agent capable of antagonizing the binding of cortisol to a glucocorticoid receptor is useful in methods for treating a patient diagnosed with Amyotrophic Lateral Sclerosis (ALS).

### BACKGROUND OF THE INVENTION

Amyotrophic lateral sclerosis (ALS) is a rapidly progressive neurodegenerative disorder that is typically fatal within two to three years of clinical onset. ALS is characterized by neuronal muscle atrophy (amyotrophy) and hyperflexia due to loss of lower and upper motomeurons in the anterior horns of the spinal cord and in the corticospinal tracts, respectively. See, Mitsumoto, H. et al. Amyotrophic Lateral Sclerosis, Philadelphia, F. A. Davis, 1998. The disease affects men slightly more than women, and typically becomes clinically manifest in the fifth decade of life or later. While 90% or more of the cases are sporadic with no known etiology, 5 to 10% of the cases are familial with an autosomal dominant pattern of inheritance. For a subset of the familial cases, the genetic locus has been mapped to the copper-zinc superoxide dismutase gene (SOD1) on chromosome 21. See, Rosen, D. R., et al. (1993) Nature 362:59-62.

Currently, the only approved therapy to alter the course of the disease is the drug Riluzole, which is thought to act through the inhibition of glutamate release. The effect of Riluzole is modest, prolonging survival by about two to three months. In view of the devastating nature of the disease, and the lack of effective therapeutic approaches, a need exists for the development of improved treatment methods for patients suffering from ALS.

Notably, patients diagnosed with ALS exhibit adrenal dysregulation and loss of the circadian rhythm of cortisol levels. See, Patacchiolo et al. (2003) J. Endocrinol. Invest. 26(12):RC23-25. In particular, the salivary levels of cortisol in patients diagnosed with ALS were compared to the salivary cortisol levels from healthy control subjects. Patacchiolo et al found that the evening cortisol levels in ALS patients were significantly elevated compared to healthy controls, and that the ALS patients did not show a physiological increase in cortisol levels following an unexpected mild stress (color-word Stroop test). These results indicate a dysregulation of adrenal activity in patients with ALS. Similar elevated levels of cortisol have been observed in the wobbler mutant mouse, which is an animal model for ALS. See, Gonzalez Deniselle et al. (1997) J. Steroid Biochem Mol. Biol. 60(3-4):205-213.

Many of the actions of cortisol in the nervous system are mediated by binding to the type I (mineralocorticoid) receptor, which is preferentially occupied, relative to the type II (glucocorticoid) receptor, at physiological cortisol levels. As cortisol levels increase, more glucocorticoid receptors are occupied and activated. Because cortisol plays an essential role in metabolism, inhibition of all cortisol-mediated activities would be fatal. Therefore, antagonists that specifically prevent type II glucocorticoid receptor functions, but do not antagonize type I mineralocorticoid receptor functions are of particular use in this invention. Mifepristone and similar antagonists are examples of this category of receptor antagonists.

The present inventors have determined for the first time that glucocorticoid receptor antagonists (GRAs) such as mifepristone are effective agents for treating patients diagnosed with ALS and having normal, increased, or decreased cortisol levels. The present invention therefore fulfills a need in the art for an effective treatment for patients with ALS.

### BRIEF SUMMARY OF THE INVENTION

The invention is based in part on the discovery that administration of a glucocorticoid receptor antagonist provides an effective and improved treatment for patients diagnosed with amyotrophic lateral sclerosis. Thus, one aspect of the invention is directed towards methods of treating a patient diagnosed with ALS by administering a therapeutically effective amount of a glucocorticoid receptor specific antagonist, provided that the subject is not otherwise in need of treatment with a glucocorticoid receptor antagonist.

In some embodiments of the invention, the glucocorticoid receptor antagonist comprises a steroid compound. In some embodiments, the glucocorticoid receptor antagonist comprises a steroidal skeleton with at least one phenyl-containing moiety in the 11-β position of the steroidal skeleton. In some embodiments, the phenyl-containing moiety in the 11-β position of the steroidal skeleton is a dimethylaminophenyl moiety.

In some embodiments of the invention, the glucocorticoid receptor antagonist is mifepristone. In some embodiments of the invention, the glucocorticoid receptor antagonist is selected from the group consisting of 11β-(4-dimethylaminoethoxyphenyl)-17α-propynyl-17β-hydroxy-4,9-estradien-3-one and 17β-hydroxy-17α-19-(4-methylphenyl)androsta-4,9(11)-dien-3-one. In some embodiments, the glucocorticoid receptor antagonist is (11β,17β)-11-(1,3-benzodioxol-5-yl)-17-hydroxy-17-(1-propynyl)estra-4,9-dien-3-one.

In some embodiments, the glucocorticoid receptor antagonist includes any steroid backbone modification which effects a biological response resulting from a GR agonist interaction. Non-limiting examples of specific glucocorticoid receptor antagonist suitable for use with the present invention, and discussed in more detail below include (6β,11β,17β)-11-(4-dimethyl-aminophenyl)-6-methyl-4′,5′-dihydro[estra-4,9-diene-17,2′(3H)-furan]-3-one (“Org 31710”, see Mizutani, J Steroid Biochem Mol Biol 42(7):695-704, 1992), Org31806, Org34517, Org34116, RU43044, 17-β-hydroxy-11-β-(4-[methyl]-[1-methylethyl]aminophenyl)-17α-[prop-1-ynyl]estra-4-9-diene-3-one (“RU40555”, see Kim, J Steroid Biochem Mol. Biol. 67(3):213-22, 1998), RU28362, and ZK9829.

In some embodiments, the glucocorticoid receptor antagonist comprises a non-steroidal compound. Non-limiting exemplary non-steroidal GR antagonist compounds suitable for use with the present invention include compounds as disclosed in U.S. Pat. Pub. No. 20040176595, azadecalin and fused ring azadecalin compounds, and related compounds as disclosed in PCT/US05/08049, PCT/US05/00607, U.S. Pat. Pub Nos.: 2007/0203179 and 2007/0281928; and modified pyrimidine compounds as disclosed in PCT/US05/23675 and U.S. Pat. Pub. No. 2006/0025405.

In some embodiments, the glucocorticoid receptor antagonist comprises a non-steroidal compound, with the proviso that the compound is not a tricyclic compound. In some embodiments, the glucocorticoid receptor antagonist comprises a non-steroidal compound, with the proviso that the compound is not (4β,7R,8αR)-4β-benzyl-7-hydroxy-N-(2-methylpyridin-3-yl)-7-(trifluoromethyl)-4β,5,6,7,8,8α,9,10-octahydrophenanthrene-2-carboxamide or (2R,4αS,10αR)-4α-benzyl-7-((2-methylpyridin-3-yl)carbamoyl)-2-(trifluoromethyl)-1,2,3,4,4α,9,10,10α-octahydrophenanthren-2-yl dihydrogen phosphate wherein R is —H or —P(O)(OH)₂, or a salt thereof. In some embodiments the glucocorticoid receptor antagonist comprises a non-steroidal compound, with the proviso that the compound is not selected from the group consisting of (4βS,7S,8αR)-4β-benzyl-7-hydroxy-N-((2-methylpyridin-3-yl)-7-(3,3,3-trifluoropropyl)-4β,5,6,7,8,8α,9,10-octahydrophenanthrene-2-carboxamide; (4βS,7R,8αR)-4β-benzyl-N-(3,5-dimethylpyrazin-2-yl)-7-hydroxy-7-(trifluoromethyl)-4β,5,6,7,8,8α,9,10-octahydrophenanthrene-2-carboxamide; (4βS,7S,8αR)-4β-benzyl-7-hydroxy-N-(2-methylpyridin-3-yl)-7-(3,3,3-trifluoropropyl)-4β,5,6,7,8,8α,9,10-octahydrophenanthrene-2-carboxamide; (4βS,7R,8αR)-4β-benzyl-7-hydroxy-N-(4-methylpyridin-3-yl)-7-(trifluoropropyl)-4β,5,6,7,8,8α, 9,10-octahydrophenanthrene-2-carboxamide; (4βS,7R,8αS)-4β-benzyl-7-hydroxy-N-(2-methylpyridin-3-yl)-10-oxo-7-(trifluoromethyl)-4β,5,6,7,8,8α,9,10-octahydrophenanthrene-2-carboxamide; (4βS,7R,8αR,10R)-4β-benzyl-7,10-dihydroxy-N-(2-methylpyridin-3-yl)-7-(trifluoromethyl)-4β,5,6,7,8,8α,9,10-octahydrophenanthrene-2-carboxamide; (4βS,7R,8αR)-4β-benzyl-7-(difluoromethyl)-7-hydroxy-N-(2-methylpyridin-3-yl)-4β,5,6,7,8,8α, 9,10-octahydrophenanthrene-2-carboxamide; (4βS,7R,8αS)-4β-benzyl-7-hydroxy-N-(2-methylpyridin-3-yl)-7-(trifluoromethyl)-4β,5,6,7,8,8α-hexahydrophenanthrene-2-carboxamide; (4βS,7S,8αR)-4β-benzyl-N-(2,4-dimethylpyrimidin-5-yl)-7-hydroxy-7-(3,3,3-trifluoropropyl)-4β,5,6,7,8,8α, 9,10-octahydrophenanthrene-2-carboxamide; and (2R,4αS,10αR)-4α-benzyl-7-((2-methylpyridin-3-yl)carbamoyl)-2-(trifluoromethyl)-1,2,3,4,4α,9,10,10α-octahydrophenanthren-2-yl isobutyl carbonate.

In some embodiments, the gluococorticoid receptor antagonist comprises a non-steroidal compound with the proviso that the compound does not have the structure:

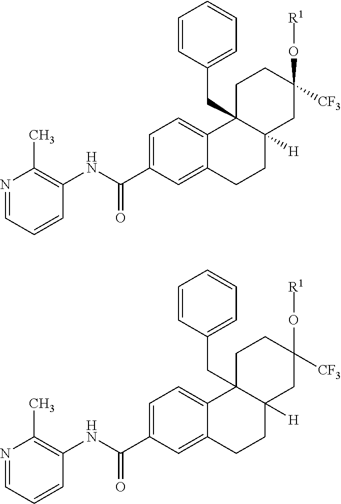

Wherein R¹ is —H or —P(O)(OH)₂; or a salt thereof.

In some embodiments, the gluococorticoid receptor antagonist is used to treat patients having a sporadic form of ALS. In some embodiments, the gluococorticoid receptor antagonist is used to treat patients having a familial form of ALS. In some embodiments, the gluococorticoid receptor antagonist is used to treat patients having a form of ALS that predominantly affects the lower motorneurons (e.g. progressive muscular atrophy). In some embodiments, the gluococorticoid receptor antagonist compound is used to treat patients having a form of ALS that predominantly affects the lower brainstem cranial motor nuclei (e.g., progressive bulbar palsy and bulbar amyotrophic lateral sclerosis).

In some embodiments, the glucocorticoid receptor antagonist is administered in a daily amount of between about 0.5 to about 40 mg per kilogram of body weight per day, preferably between about 5 to about 20 mg per kilogram of body weight per day. In some embodiments, the GRA is administered in an amount of between about 1 to about 4 mg per kilogram of body weight per day. The invention further provides for methods where the GRA is administered twice a day, once a day, once every other day, twice a week, or once a week. In some embodiments the GRA is administered by mouth (orally), by transdermal application, by a nebulized suspension, by an aerosol spray, by injection, or by an intraocular, intrathecal, intravaginal, or intrarectal route, including suppositories.

### DETAILED DESCRIPTION OF THE INVENTION

### I. Definitions

The term “amyotrophic lateral sclerosis” or “ALS” as used herein refers to the group of neurodegenerative diseases characterized by the loss of motorneurons in the ventral horn of the spinal cord and the cortical neurons that provide their afferent input. ALS includes both the sporadic and familial forms, as well as forms that predominantly affect either the lower motorneurons (e.g., progressive muscular atrophy) and forms that predominantly affect the lower brainstem cranial motor nuclei (e.g., progressive bulbar palsy and bulbar amyotrophic lateral sclerosis).

The phrase “ameliorating the symptoms” or “palliative treatment” and the corresponding terms “treat” and “treatment” refers to treatment that eases or reduces the effect or intensity of a symptom of ALS, without curing the disease. Any indicia of success in alleviating or reducing the symptoms of ALS is recognized as ameliorating the symptoms, or providing palliative treatment. The prevention or reduction of ALS symptoms can be determined using standard routine clinical tests and observations well within the skill and knowledge of a medical professional. Non-limiting exemplary tests can include imaging tests, such as magnetic resonance imaging (MRI) or contrast myelography; neurophysiology tests, including electromyography tests and nerve conduction velocity tests; as well as observations made during a physical examination can each be used to assess the success of a GRA in inhibiting or ameliorating the symptoms associated with ALS and/or slowing the rate of disease progression.

The term “cortisol” refers to a family of compositions also referred to as hydrocortisone, and any synthetic or natural analogues thereof.

The term “glucocorticoid receptor” (“GR”) refers to a family of receptors also referred to as the cortisol receptor, which specifically binds to cortisol and/or cortisol analogs. The term includes isoforms of GR, recombinant GR and mutated GR.

The term “glucocorticoid receptor specific antagonist” refers to any composition or compound that partially or completely inhibits (antagonizes) the binding of a glucocorticoid receptor (GR) agonist, such as cortisol, or cortisol analogs, synthetic or natural, to a GR. By “specific”, we intend the drug to preferentially bind to the GR rather than the mineralocorticoid receptor (MR) with an affinity of at least 100-fold, and frequently 1000-fold.

The term “mifepristone” refers to a family of compositions that include RU486, 17β-hydroxy-11β-(4-dimethyl-aminophenyl)-17α-(1-propynyl)-estra-4,9-dien-3-one), 11β-(4dimethylaminophenyl)-17β-hydroxy-17α-(1-propynyl)-estra-4,9-dien-3-one), and analogs thereof, which bind to a GR, typically with high affinity, and antagonize the binding of a cortisol or a cortisol analogue to the GR. Chemical names for RU-486 vary; for example, RU486 has also been termed: 11β-[p-(dimethylamino)phenyl]-17β-hydroxy-17-(1-propynyl)-estra-4,9-dien-3-one; 11β-(4-dimethyl-aminophenyl)-17β-hydroxy-17α-(prop-1-ynyl)-estra-4,9-dien-3-one; 17β-hydroxy-11β-(4-dimethylaminophenyl-1)-17α-(propynyl-1)-estra-4,9-diene-3-one; 17β-hydroxy-11β-(4-dimethylaminophenyl-1)-17α-(propynyl-1)-E; (11β,17β)-11-[4-dimethylamino)-phenyl]-17-hydroxy-17-(1-propynyl)estra-4,9-dien-3-one; and 11β-[4-(N,N-dimethylamino)phenyl]-17α-(prop-1-ynyl)-D-4,9-estradiene-17β-ol-3-one. Additional names and compounds are well known to persons of skill in the art.

A patient “not otherwise in need of treatment with a glucocorticoid receptor antagonist” is a patient who is not suffering from a condition known in the art to be effectively treatable with glucocorticoid receptor antagonists. Conditions known in the art to be effectively treatable with glucocorticoid receptor antagonists can include, but are not limited to: Cushing's disease, drug withdrawal, psychosis, dementia, stress disorders, and psychotic major depression.

Where substituent groups are specified by their conventional chemical formulae, written from left to right, they equally encompass the chemically identical substituents that would result from writing the structure from right to left, e.g., —CH2O— is equivalent to OCH2-.

### II. Introduction

The invention pertains to the surprising discovery that agents capable of antagonizing the binding of cortisol (or any synthetic or natural analogue thereof) to a GR is effective in the treatment of patients diagnosed with amyotrophic lateral sclerosis (ALS). In one embodiment, the methods of the invention use agents that act as glucocorticoid receptor antagonist (GRA) antagonists, to ameliorate the symptoms of ALS or slow the progression of the disease. The methods of the invention are effective in treating patients with ALS having normal, increased or decreased levels of cortisol, or other natural or synthetic glucocorticoids.

Cortisol acts by binding to a glucocorticoid receptor (GR). In humans, glucocorticoid receptors are thought to be present in at least two forms: a GR-α form of 777 amino acids; and, a GR-β isoform that differs in the carboxy-terminal fifteen amino acids. The two types of GR have high affinity for their specific ligands, and are considered to function through the same signal transduction pathways.

The biologic effects of cortisol, including pathologies or dysfunctions caused by hypercortisolemia, can be modulated and controlled at the GR level using receptor antagonists. Several different classes of agents are able to act as GR antagonists, i.e., to block the physiologic effects of GR-agonist binding (the natural agonist is cortisol). These antagonists include compositions, which by binding to GR, block the ability of an agonist to effectively bind to and/or activate the GR. One family of known GR antagonists, mifepristone and related compounds, are effective and potent anti-glucocorticoid agents in humans (Bertagna, J. Clin. Endocrinol. Metab. 59:25, 1984). Mifepristone binds to the GR with high affinity, with a K of dissociation<10⁻⁹ M (Cadepond, Annu. Rev. Med. 48:129, 1997). Thus, in one embodiment of the invention, mifepristone and related compounds are used to treat patients diagnosed with ALS to ameliorate the symptoms and/or slow the progression of the disease.

The methods of the invention include use of GRAS to inhibit the biological effects of an agonist-bound GR, illustrative compounds and compositions which can be used in the treatment of patients diagnosed with ALS are set forth. Routine procedures that can be used to identify further compounds and compositions suitable for use in practicing the methods of the invention are also described. As the invention provides for administering these compounds and compositions as pharmaceuticals, routine means to determine GRA drug regimens and formulations to practice the methods of the invention are set forth below.

### III. Diagnosis and Monitoring of an ALS Patient

Because there is no known cure for ALS, it is imperative that potentially remediable causes of motorneuron dysfunction be excluded during the diagnosis of ALS. This is particularly important for cases that are atypical by virtue of (1) predominantly affecting either upper or lower motorneurons; (2) involvement of neurons other than motorneurons; and (3) evidence of motorneuronal conduction block on electrophysiologic testing. Absence of pain or of sensory changes, normal bowel and bladder function, normal roentgenographic studies of the spine and normal cerebrospinal fluid (CSF) all favor a diagnosis of ALS. Where doubt exists, a magnetic resonance imaging (MRI) scan and contrast myelography studies should be performed to visualize the cervical spinal cord.

Early symptoms of ALS includes asymmetric weakness of the hands, typically manifest as dropping objects and difficulty performing fine motor tasks, and cramping and spasticity of the arms and legs. As the disease progresses, muscle strength and bulk diminish and involuntary contractions of individual motor units (i.e. fasciculations) occur. The disease eventually progresses to involve the respiratory muscles leading to recurrent bouts of pulmonary infection. The degree of severity of involvement of the upper and motorneurons is variable, and the term progressive muscular atrophy is applied to the relatively uncommon cases where lower motorneuron involvement predominates. In some patients degeneration of the lower brainstem cranial motor nuclei occurs early and progresses rapidly. These patients are referred to as having progressive bulbar palsy or bulbar amyotrophic lateral sclerosis. In these patients abnormalities of deglutition and phonation dominate. In patients having familial ALS, the symptoms typically appear earlier than in the sporadic cases, but the clinical course is comparable.

### IV. General Laboratory Procedures

When practicing the methods of the invention, a number of general laboratory tests can be used to assist in the progress of the patient under AD administration, including monitoring of parameters such as blood cortisol, drug metabolism, etc. These procedures can be helpful because all patients metabolize and react to drugs uniquely. In addition, such monitoring may be important because each GR antagonist has different pharmacokinetics. Different patients and AD medications may require different dosage regimens and formulations. Such procedures and means to determine dosage regimens and formulations are well described in the scientific and patent literature. A few illustrative examples are set forth below.

A. Determining Blood Cortisol Levels

Varying levels of blood cortisol have been associated with patients having ALS, however, the invention may also be practiced upon patients with apparently normal, or even reduced levels of blood cortisol. Thus, monitoring blood cortisol and determining baseline cortisol levels are useful laboratory tests to aid in monitoring the symptoms, and the rate of disease progression in patients diagnosed with ALS and being treated with the methods of the invention. A wide variety of laboratory tests exist that can be used to determine whether an individual is normal, hypo- or hypercortisolemic.

Immunoassays such as radioimmunoassays are commonly used because they are accurate, easy to do and relatively cheap. Because levels of circulating cortisol are an indicator of adrenocortical function, a variety of stimulation and suppression tests, such as ACTH Stimulation, ACTH Reserve, or dexamethasone suppression (see, e.g., Greenwald, Am. J. Psychiatry 143:442-446, 1986), can also provide diagnostic, prognostic or other information to be used adjunctively in the methods of the invention.

One such assay available in kit form is the radioimmunoassay available as “Double Antibody Cortisol Kit” (Diagnostic Products Corporation, Los Angeles, Calif.), (Acta Psychiatr. Scand. 70:239-247, 1984). This test is a competitive radioimmunoassay in which ¹²⁵I-labeled cortisol competes with cortisol from an clinical sample for antibody sites. In this test, due to the specificity of the antibody and lack of any significant protein effect, serum and plasma samples require neither pre-extraction nor pre-dilution.

B. Determination of Blood/Urine Mifepristone Levels

Because a patient's metabolism, clearance rate, toxicity levels, etc. differs with variations in underlying primary or secondary disease conditions, drug history, age, general medical condition and the like, it may be necessary to measure blood and urine levels of GR antagonist. Means for such monitoring are well described in the scientific and patent literature. As in one embodiment of the invention mifepristone is administered to ameliorate the symptoms and/or slow the progression of the disease, an illustrative example of determining blood and urine mifepristone levels is set forth in the Example below.

C. Other Laboratory Procedures

Because the mechanism underlying neurodegeneration in patients with ALS may be complex, a number of additional laboratory tests can be used adjunctively in the methods of the invention to assist in diagnosis, treatment efficacy, prognosis, toxicity and the like. For example, diagnosis and treatment assessment can be augmented by monitoring and measuring glucocorticoid-sensitive variables, including but limited to fasting blood sugar, blood sugar after oral glucose administration, plasma concentrations thyroid stimulating hormone (TSH), corticosteroid-binding globulin, luteinizing hormone (LH), testosterone-estradiol-binding globulin, leptin, insulin, and/or total and free testosterone.

Laboratory tests monitoring and measuring GR antagonist metabolite generation, plasma concentrations and clearance rates, including urine concentration of antagonist and metabolites, may also be useful in practicing the methods of the invention. For example, mifepristone has two hydrophilic, N-monomethylated and N-dimethylated, metabolites. Plasma and urine concentrations of these metabolites (in addition to RU486) can be determined using, for example, thin layer chromatography, as described in Kawai, Pharmacol. and Experimental Therapeutics 241:401-406, 1987.

D. Non-Limiting Exemplary GRAs Suitable for Use with the Invention

The invention provides for methods of ameliorating the symptoms and/or slowing disease progression in patients with ALS utilizing any composition or compound that can antagonize the binding of cortisol or a cortisol analogue to a GR. Antagonists of GR activity utilized in the methods of the invention are well described in the scientific and patent literature. An illustrative example is set forth below.

1. Steroidal Anti-Glucocorticoids as GR Antagonists.

Steroidal glucocorticoid antagonists are administered to ameliorate the symptoms and/or slow disease progression in patients diagnosed with ALS in various embodiments of the invention. Steroidal antiglucocorticoids can be obtained by modification of the basic structure of glucocorticoid agonists, i.e., varied forms of the steroid backbone. The structure of cortisol can be modified in a variety of ways. The two most commonly known classes of structural modifications of the cortisol steroid backbone to create glucocorticoid antagonists include modifications of the 11-beta hydroxy group and modification of the 17-beta side chain (see, e.g., Lefebvre, J. Steroid Biochem. 33:557-563, 1989).

Examples of steroidal GR antagonists include androgen-type steroid compounds as described in U.S. Pat. No. 5,929,058, and the compounds disclosed in U.S. Pat. Nos. 4,296,206; 4,386,085; 4,447,424; 4,477,445; 4,519,946; 4,540,686; 4,547,493; 4,634,695; 4,634,696; 4,753,932; 4,774,236; 4,808,710; 4,814,327; 4,829,060; 4,861,763; 4,912,097; 4,921,638; 4,943,566; 4,954,490; 4,978,657; 5,006,518; 5,043,332; 5,064,822; 5,073,548; 5,089,488; 5,089,635; 5,093,507; 5,095,010; 5,095,129; 5,132,299; 5,166,146; 5,166,199; 5,173,405; 5,276,023; 5,380,839; 5,348,729; 5,426,102; 5,439,913; 5,616,458, 5,696,127, and 6,303,591. Such steroidal GR antagonists include cortexolone, dexamethasone-oxetanone, 19-nordeoxycorticosterone, 19-norprogesterone, cortisol-21-mesylate; dexamethasone-21-mesylate, 11β-(4-dimethylaminoethoxyphenyl)-17α-propynyl-17β-hydroxy-4,9estradien-3-one (RU009), and 17β-hydroxy-17α-19-(4-methylphenyl)androsta-4,9(11)-dien-3-one (RU044).

Other examples of steroidal antiglucocorticoids are disclosed in Van Kampen et al. (2002) Eur. J. Pharmacol. 457(2-3):207; PCT Int'l Pub. No. WO 03/043640 and; European Pat. App. Nos. EP 0 683 172 B1, and EP 0 763 541 B1, each of which is herein incorporated by reference. Furthermore, EP 0 763 541 and Hoyberg et al., Int'l J. Neuro-psychopharmacology, Suppl. 1, 5148 (2002); discloses the compound (11β,17β)-11-(1,3-benzodioxol-5-yl)-17-hydroxy-17-(1-propynyl)estra-4,9-dien-3-one (ORG 34517), which in some embodiments is administered in an amount effective to ameliorate or inhibit ALS disease symptoms, and/or slow the rate of disease progression in a patient diagnosed with ALS.

a) Removal or Substitution of the 11-Beta Hydroxy Group

Glucocorticoid agonists with modified steroidal backbones comprising removal or substitution of the 11-beta hydroxy group are administered in one embodiment of the invention. This class includes natural antiglucocorticoids, including cortexolone, progesterone and testosterone derivatives, and synthetic compositions, such as mifepristone (Lefebvre, et al. (1989)). Preferred embodiments of the invention include all 11-beta-aryl steroid backbone derivatives because these compounds are devoid of progesterone receptor (PR) binding activity (Agarwal, FEBS 217:221-226, 1987). Another preferred embodiment comprises an 11-beta phenyl-aminodimethyl steroid backbone derivative, i.e., mifepristone, which is both an effective anti-glucocorticoid and anti-progesterone agent. These compositions act as reversibly-binding steroid receptor antagonists. For example, when bound to a 11-beta phenyl-aminodimethyl steroid, the steroid receptor is maintained in a conformation that cannot bind its natural ligand, such as cortisol in the case of GR (Cadepond, (1997)).

Synthetic 11-beta phenyl-aminodimethyl steroids include mifepristone, also known as RU486, or 17β-hydroxy-11β-(4-dimethyl-aminophenyl)17α-(1-propynyl)estra-4,9-dien-3-one). Mifepristone has been shown to be a powerful antagonist of both the progesterone and glucocorticoid (GR) receptors. Another 11β phenyl-aminodimethyl steroid shown to have GR antagonist effects includes RU009 (RU39.009), 11β-(4-dimethyl-aminoethoxyphenyl)-17α-(propynyl-17β-hydroxy-4,9-estradien-3-one). See, Bocquel, J. Steroid Biochem. Molec. Biol. 45:205-215, 1993. Another GR antagonist related to RU486 is RU044 (RU43.044) 17β-hydroxy-17α-19-(4-methyl-phenyl)-androsta-4,9(11)-dien-3-one (Bocquel, (1993)). See also, Teutsch, Steroids 38:651-665, 1981; U.S. Pat. Nos. 4,386,085 and 4,912,097.

One embodiment includes compositions containing the basic glucocorticoid steroid structure that are irreversible anti-glucocorticoids. Such compounds include alpha-keto-methane-sulfonate derivatives of cortisol, including cortisol-21-mesylate(4-pregnene-11β,17α,21-triol-3,20-dione-21-methane-sulfonate) and dexamethasone-21-mesylate(16-methyl-9α-fluoro-1,4-pregnadiene-11β,17α,21-triol-3,20-dione-21-methane-sulfonate). See, Simons, J. Steroid Biochem. 24:25-32, 1986; Mercier, J. Steroid Biochem. 25:11-20, 1986; U.S. Pat. No. 4,296,206.

b) Modification of the 17β Side Chain Group

Steroidal antiglucocorticoids which can be obtained by various structural modifications of the 17-β side chain are also used in the methods of the invention. This class includes synthetic antiglucocorticoids such as dexamethasone-oxetanone, various 17,21-acetonide derivatives and 17-beta-carboxamide derivatives of dexamethasone (Lefebvre, 1989; Rousseau, Nature 279:158-160, 1979).

c) Other Steroid Backbone Modifications

GR antagonists used in the various embodiments of the invention include any steroid backbone modification which effects a biological response resulting from a GR-agonist interaction. Steroid backbone antagonists can be any natural or synthetic variation of cortisol, such as adrenal steroids missing the C-19 methyl group, such as 19-nordeoxycorticosterone and 19-norprogesterone (Wynne, Endocrinology 107:1278-1280, 1980).

In general, the 11β side chain substituent, and particularly the size of that substituent, can play a key role in determining the extent of a steroid's antiglucocorticoid activity. Substitutions in the A ring of the steroid backbone can also be important. 17-hydroxypropenyl side chains generally decrease antiglucocorticoid activity in comparison to 17-propinyl side chain containing compounds.

Additional glucocorticoid receptor antagonists known in the art and suitable for practice of the invention include 21-hydroxy-6,19-oxidoprogesterone (see, Vicent, Mol. Pharm. 52:749-753, 1997); ORG 31710, (6β,11β,17β)-11-(4-(dimethyl-amino)phenyl)-6-methyl-4′,5′-dihydro[estra-4,9-diene-17,2′(3H)-furan]-3-one, (see, Mizutani, J Steroid Biochem Mol Biol 42(7):695-704, 1992); ORG 34517, (11β,17β)-11-(1,3-benzodioxol-5-yl)-17-hydroxy-17-(1-propynyl)e-stra-4,9-dien-3-one, as disclosed in Hoyberg et al., Int'l J. of Neuro-psychopharmacology, 5: Supp. 1, S148 (2002); ORG 33628, [(11β,17α)-11-(4-acetylphenyl)-17,23-epoxy-19,24-dinorchola-4,-9,20-trien-3-one]; ORG 31806, [(7β,11β,17β)-11-(4-(dimethylamino)phenyl)-7-Me-4′,5′-dihydrospiro(oestra-4,9-diene-17,2′(3′H)-furan)-3-one]-; ORG 34116, (11β,17α)-11,21-Bis[4-(dimethylamino)phenyl]-17-hydroxy-19-norpregna-4,9,dien-20-yn-3-one; ORG 34850, (11β,17α)-11-[4-(dimethylamino)phenyl]-17-hydroxy-21-[4-(methylsulfonyl)phenyl-19-norpregna-4,9-dien-20-yn-3-one, and related compounds disclosed in U.S. Pat. No. 5,741,787; RU43044, (17β-hydroxy-11β-4-[methyl]-[1-methylethyl]aminophenyl/-17α-[prop-1-ynyl]estra-4-9-diene-3-one)“RU40555”, see Kim, J Steroid Biochem Mol. Biol. 67(3):213-22, 1998), RU28362, and ZK98299.

2. Non-Steroidal Anti-Glucocorticoids as Antagonists.

Non-steroidal glucocorticoid antagonists are also used in the methods of the invention to ameliorate the symptoms of and/or slow the progression of the disease in patients diagnosed with ALS. These include synthetic mimetics and analogs of proteins, including partially peptidic, pseudopeptidic and non-peptidic molecular entities. For example, oligomeric peptidomimetics useful in the invention include (α-β-unsaturated) peptidosulfonamides, N-substituted glycine derivatives, oligo carbamates, oligo urea peptidomimetics, hydrazinopeptides, oligosulfones and the like (see, e.g., Amour, Int. J. Pept. Protein Res. 43:297-304, 1994; de Bont, Bioorganic & Medicinal Chem. 4:667-672, 1996). The creation and simultaneous screening of large libraries of synthetic molecules can be carried out using well-known techniques in combinatorial chemistry, for example, see van Breemen, Anal Chem 69:2159-2164, 1997; and Lam, Anticancer Drug Des 12:145-167, 1997. Design of peptidomimetics specific for GR can be designed using computer programs in conjunction with combinatorial chemistry (combinatorial library) screening approaches (Murray, J. of Computer-Aided Molec. Design 9:381-395, 1995; Bohm, J. of Computer-Aided Molec. Design 10:265-272, 1996). Such “rational drug design” can help develop peptide isomerics and conformers including cycloisomers, retro-inverso isomers, retro isomers and the like (as discussed in Chorev, TibTech 13:438-445, 1995).

Examples of non-steroidal GR antagonists may include but are not limited to cis-1-acetyl-4-(4-((2-(2,4-dichlorophenyl)-2-(1H-imidazol-1-ylmethyl)-1,3-dioxolan-4-yl)methoxy)phenyl)piperazine; 1-(o-Chloro-alpha,alpha-diphenylbenzyl)imidazole; N (triphenylmethyl)imidazole; N-([2-fluoro-9-phenyl]fluorenyl)imidazole; N-([2-pyridyl]diphenylmethyl)imidazole; N (2[4,4′,4″-trichlorotrityl]oxyethyl)morpholine; 1-(2[4,4′,4″-trichlorotrityl]oxyethyl)-4 (2 hydroxyethyl)piperazine dimaleate; N-([4,4′,4″]-trichlorotrityl)imidazole; 9-(3-mercapto-1,2,4 triazolyl)-9-phenyl-2,7-difluorofluorenone; 1-(2-chlorotrityl)-3,5-dimethylpyrazole; 4 (morpholinomethyl)-A-(2-pyridyl)benzhydrol; 5-(5-methoxy-2-(N-methylcarbamoyl)-phenyl)dibenzosuberol; N-(2-chlorotrityl)-L-prolinol acetate; 1-(2-chlorotrityl)-2-methylimidazole; 1 (2 chlorotrityl)-1,2,4-triazole; 1,S-bis(4,4′,4″-trichlorotrityl)-1,2,4-triazole-3-thiol; and N((2,6 dichloro-3 methylphenyl)diphenyl)methylimidazole (see U.S. Pat. No. 6,051,573); the GR antagonist compounds disclosed in U.S. Pat. Nos. 5,696,127 and 6,570,020; the GR antagonist compounds disclosed in U.S. Pat. Pub. No. 20020077356, the glucocorticoid receptor antagonists disclosed in Bradley et al., J. Med. Chem. 45, 2417-2424 (2002), e.g., 4α(S)-benzyl-2(R)-chloroethynyl-1,2,3,4,4α,9,10,10α(R)-octahydrophenanthrene-2,7-diol (“CP 394531”) and 4α(S)-benzyl-2(R)-prop-1-ynyl-1,2,3,4,4α,9,10,10α(R)-octahydro-phenanthrene-2,7-diol (“CP 409069”) and related compounds disclosed in PCT Intl Pub. No. WO 00/66522; the compounds disclosed in PCT Int'l Pub. No. WO 96/19458, which describes non-steroidal compounds which are high-affinity, highly selective antagonists for steroid receptors, such as 6-substituted-1,2-dihydro-N-protected-quinolines; benzopyranol[3,4-f]quinolines described as glucocorticoid receptor modulators disclosed in PCT Int'l Pub. Nos. WO 99/41256 and WO 01/16128; aminobenzene derivatives disclosed as glucocorticoid receptor modulators disclosed in PCT Int'l Pub. No. WO 02/064550; and some opioid ligands, such as the κ opioid compounds dynorphin-1,13-diamide, U50,488 (trans-(1R,2R)-3,4-dichloro-N-methyl-N-[2-(1-pyrrolidinyl)cyclohexyl]benzeneacetamide), bremazocine and ethylketocyclazocine; and the non-specific opioid receptor ligand, naloxone, as disclosed in Evans et al., Endocrinol. 141:2294-2300 (2000); 4β(S)-benzyl-7(S)-hydroxy-7-(1-propynyl)-4β,5,6,7,8,8α(R),9,10-octahydrophenanthrene-2-carboxylic acid (pyridine-4-ylmethyl)amide (“CP-472555”), 4β(S)-benzyl-7(S)-hydroxy-7-(3,3,3-trifluoropropyl)-4β,5,6,7,8,8α(R),9,10-octahydrophenanthrene-2-carboxylic acid; (2-methylpyridin-3-ylmethyl)amide and related compounds disclosed in PCT Int'l Pub. No. WO 0066522, and in U.S. Pat. Pub. No. 20040176595; octahydrophenanthrenyl carbamates disclosed in European Pat. App. No. EP 1201649; oxadiazolylalkoxyoctahydrophenanthrenes disclosed in European Pat. App. No. EP 1201660; octahydrophenanthrene hydrazines as disclosed in PCT Int'l Pub. No. WO 2005/047254; modulators of the glucocorticoid receptor as disclosed in PCT Int'l Pub. No. WO 04/005299; tricyclic compounds disclosed in PCT Int'l Pub. Nos. WO 05/011336 and WO 05/011337; Wieland-Miescher ketone derivatives disclosed in PCT Int'l Pub. No. WO 03/011755; cyclopent[f]indazole and benz[f]indazole derivatives disclosed in PCT Int'l. Pub. No. WO 04/075840; spirocyclic compounds disclosed in PCT Int'l Pub. No. WO 04/093805; octahydro-2-H-naphtho[1,2,-f]indole-4-carboxamide derivatives disclosed in PCT Int'l. Pub. No. WO 2004/026248; cholic acid derivatives disclosed in PCT Int'l. Pub. No. WO 04/000869; dibenzopyran derivatives disclosed in PCT int'l. Pub. No. WO 01/16128; 6H-dibenzo[b,d]pyran derivatives disclosed in U.S. Pat. Pub. Nos. 20020049322 and 20030220332; substituted aminobenzene derivatives disclosed in PCT Int'l. Pub. No. WO 02/064550; triphenylmethane derivatives disclosed in U.S. Pat. No. 6,166,013; the compound (3,5-dibromo-4-[5-isopropyl-4-methoxy-2-(3-methylbenzoyl-phenoxy]phenyl)acetic acid (“KB285”) disclosed in PCT Int'l. Pub. No. WO 99/63976 and related compounds disclosed in PCT Int'l Pub. Nos. WO 01/047859, WO 02/43648 and WO 02/44120; azadecalin derivatives disclosed in PCT Int'l Pub. No. WO 05/070893 and U.S. patent application Ser. No. 10/596,998; fused ring azadecalin compounds disclosed in PCT Int'l Pub. No. WO 05/087769 and U.S. patent application Ser. No. 10/591,884; modified pyrimidine compounds disclosed in PCT/US05/23675 and U.S. patent application Ser. No. 11/174,096.

E. Identifying Specific Glucocorticoid Receptor Antagonists

Because any specific GR antagonist can be used to ameliorate the symptoms and/or slow progression of the disease in patients diagnosed with ALS in the methods of the invention, in addition to the compounds and compositions described above, additional useful GR antagonists can be determined by the skilled artisan. A variety of such routine, well-known methods can be used and are described in the scientific and patent literature. They include in vitro and in vivo assays for the identification of additional GR antagonists. A few illustrative examples are described below.

One assay that can be used to identify a GR antagonist of the invention measures the effect of a putative GR antagonist on tyrosine amino-transferase activity in accordance with the method of Granner, Meth. Enzymol. 15:633, 1970. This analysis is based on measurement of the activity of the liver enzyme tyrosine amino-transferase (TAT) in cultures of rat hepatoma cells (RHC). TAT catalyzes the first step in the metabolism of tyrosine and is induced by glucocorticoids (cortisol) both in liver and hepatoma cells. This activity is easily measured in cell extracts. TAT converts the amino group of tyrosine to 2-oxoglutaric acid. β-hydroxyphenylpyruvate is also formed. It can be converted to the more stable p-hydroxybenzaldehyde in an alkaline solution and quantitated by absorbance at 331 nm. The putative GR antagonist is co-administered with cortisol to whole liver, in vivo or ex vivo, or hepatoma cells or cell extracts. A compound is identified as a GR antagonist when its administration decreases the amount of induced TAT activity, as compared to control (i.e., only cortisol or GR agonist added) (see also Shirwany, Biochem. Biophys. Acta 886:162-168, 1986).

Further illustrative of the many assays which can be used to identify compositions utilized in the methods of the invention, in addition to the TAT assay, are assays based on glucocorticoid activities in vivo. For example, assays that assess the ability of a putative GR antagonist to inhibit uptake of ³H-thymidine into DNA in cells which are stimulated by glucocorticoids can be used. Alternatively, the putative GR antagonist can complete with ³H-dexamethasone for binding to a hepatoma tissue culture GR (see, e.g., Choi, et al., Steroids 57:313-318, 1992). As another example, the ability of a putative GR antagonist to block nuclear binding of ³H-dexamethasone-GR complex can be used (Alexandrova et al., J. Steroid Biochem. Mol. Biol. 41:723-725, 1992). To further identify putative GR antagonists, kinetic assays able to discriminate between glucocorticoid agonists and antagonists by means of receptor-binding kinetics can also be used (as described in Jones, Biochem J. 204:721-729, 1982).

In another illustrative example, the assay described by Daune, Molec. Pharm. 13:948-955, 1977; and in U.S. Pat. No. 4,386,085, can be used to identify anti-glucocorticoid activity. Briefly, the thymocytes of adrenalectomized rats are incubated in nutritive medium containing dexamethasone with the test compound (the putative GR antagonist) at varying concentrations. ³H-uridine is added to the cell culture, which is further incubated, and the extent of incorporation of radiolabel into polynucleotide is measured. Glucocorticoid agonists decrease the amount of ³H-uridine incorporated. Thus, a GR antagonist will oppose this effect.

For additional compounds that can be utilized in the methods of the invention and methods of identifying and making such compounds, see U.S. Pat. Nos. 4,296,206 (see above); 4,386,085 (see above); 4,447,424; 4,477,445; 4,519,946; 4,540,686; 4,547,493; 4,634,695; 4,634,696; 4,753,932; 4,774,236; 4,808,710; 4,814,327; 4,829,060; 4,861,763; 4,912,097; 4,921,638; 4,943,566; 4,954,490; 4,978,657; 5,006,518; 5,043,332; 5,064,822; 5,073,548; 5,089,488; 5,089,635; 5,093,507; 5,095,010; 5,095,129; 5,132,299; 5,166,146; 5,166,199; 5,173,405; 5,276,023; 5,380,839; 5,348,729; 5,426,102; 5,439,913; and 5,616,458; U.S. Pat. App. 20040176595, and WO 96/19458, which describes non-steroidal compounds which are high-affinity, highly selective modulators (antagonists) for steroid receptors, such as 6-substituted-1,2-dihydro N−1 protected quinolines.

The specificity of the antagonist for the GR relative to the MR can be measured using a variety of assays known to those of skill in the art. For example, specific antagonists can be identified by measuring the ability of the antagonist to bind to the GR compared to the MR (see, e.g., U.S. Pat. Nos. 5,606,021; 5,696,127; 5,215,916; 5,071,773). Such an analysis can be performed using either direct binding assay or by assessing competitive binding to the purified GR or MR in the presence of a known antagonist. In an exemplary assay, cells that are stably expressing the glucocorticoid receptor or mineralocorticoid receptor (see, e.g., U.S. Pat. No. 5,606,021) at high levels are used as a source of purified receptor. The affinity of the antagonist for the receptor is then directly measured. Those antagonists that exhibit at least a 100-fold higher affinity, often 1000-fold, for the GR relative to the MR are then selected for use in the methods of the invention.

A GR-specific antagonist may also be defined as a compound that has the ability to inhibit GR-mediated activities, but not MR-mediated activities. One method of identifying such a GR-specific antagonist is to assess the ability of an antagonist to prevent activation of reporter constructs using transfection assays (see, e.g., Bocquel et al, J. Steroid Biochem Molec. Biol. 45:205-215, 1993; U.S. Pat. Nos. 5,606,021, 5,929,058). In an exemplary transfection assay, an expression plasmid encoding the receptor and a reporter plasmid containing a reporter gene linked to receptor-specific regulatory elements are co-transfected into suitable receptor-negative host cells. The transfected host cells are then cultured in the presence and absence of a hormone, such as cortisol or an analog thereof, able to activate the hormone responsive promoter/enhancer element of the reporter plasmid. Next the transfected and cultured host cells are monitored for induction (i.e., the presence) of the product of the reporter gene sequence. Finally, the expression and/or steroid binding-capacity of the hormone receptor protein (coded for by the receptor DNA sequence on the expression plasmid and produced in the transfected and cultured host cells), is measured by determining the activity of the reporter gene in the presence and absence of an antagonist. The antagonist activity of a compound may be determined in comparison to known antagonists of the GR and MR receptors (see, e.g., U.S. Pat. No. 5,696,127). Efficacy is then reported as the percent maximal response observed for each compound relative to a reference antagonist compound. A GR-specific antagonist is considered to exhibit at least a 100-fold, often 1000-fold or greater, activity towards the GR relative to the MR.

### V. Pharmaceutical Formulations and Dosages

Antiglucocorticoids, such as mifepristone, are formulated as pharmaceuticals to be used in the methods of the invention to treat patients diagnosed with ALS. Any composition or compound that antagonizes the binding of an agonist to a GR can be used as a pharmaceutical in the invention. Routine means to determine GR antagonist drug regimens and formulations to practice the methods of the invention are well described in the patent and scientific literature, and some illustrative examples are set forth below.

A. Formulations

The GR antagonists used in the methods of the invention can be administered by any means known in the art, e.g., parenterally, topically, orally, or by local administration, such as by aerosol or transdermally. The GR antagonists as pharmaceutical formulations can be administered in a variety of unit dosage forms depending upon the condition or disease and the degree of severity, the general medical condition of each patient, the resulting preferred method of administration and the like. Details on techniques for formulation and administration are well described in the scientific and patent literature, See, e.g., Remington's Pharmaceutical Sciences, Maack Publishing Co, Easton Pa. (“Remington's”). Therapeutically effective amounts of glucocorticoid blockers suitable for practice of the method of the invention may range from about 0.5 to about 25 milligrams per kilogram (mg/kg). A person of ordinary skill in the art will be able without undue experimentation, having regard to that skill and this disclosure, to determine a therapeutically effective amount of a particular glucocorticoid blocker compound for practice of this invention.

In general, glucocorticoid blocker compounds may be administered as pharmaceutical compositions by any method known in the art for administering therapeutic drugs. Compositions may take the form of tablets, pills, capsules, semisolids, powders, sustained release formulations, solutions, suspensions, elixirs, aerosols, or any other appropriate compositions; and comprise at least one compound of this invention in combination with at least one pharmaceutically acceptable excipient. Suitable excipients are well known to persons of ordinary skill in the art, and they, and the methods of formulating the compositions, may be found in such standard references as Remington's Pharmaceutical Sciences, 17th ed., Mack Publishing Company, Easton Pa., 1985. Suitable non-limiting exemplary liquid carriers can include water, aqueous saline solution, aqueous dextrose solution, and glycols.

Aqueous suspensions of the invention contain a GR antagonist in admixture with one or more excipients suitable for the manufacture of aqueous suspensions. Non-limiting exemplary excipients can include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil suspensions can be formulated by suspending a GR antagonist in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

Glucocorticoid blocker pharmaceutical formulations can be prepared according to any method known to the art for the manufacture of pharmaceuticals. Such drugs can contain sweetening agents, flavoring agents, coloring agents and preserving agents. Any glucocorticoid blocker formulation can be admixtured with nontoxic pharmaceutically acceptable excipients, which are suitable for manufacture.

Typically, glucocorticoid blocker compounds suitable for use in the practice of this invention will be administered orally. The amount of a compound of this invention in the composition may vary widely depending on the type of composition, size of a unit dosage, kind of excipients, and other factors well known to those of ordinary skill in the art. In general, the final composition may comprise from 0.000001 percent by weight (% w) to 10% w of the glucocorticoid blocker compounds, preferably 0.00001% w to 1% w, with the remainder being the excipient or excipients. For example, the GR antagonist mifepristone is given orally in tablet form, with dosages in the range of between about 0.5 and 25 mg/kg, more preferably between about 0.75 mg/kg and 15 mg/kg, most preferably about 10 mg/kg.

Pharmaceutical formulations for oral administration can be formulated using pharmaceutically acceptable carriers well known in the art in dosages suitable for oral administration. Such carriers enable the pharmaceutical formulations to be formulated in unit dosage forms as tablets, pills, powder, dragees, capsules, liquids, lozenges, gels, syrups, slurries, suspensions, etc. suitable for ingestion by the patient. Pharmaceutical preparations for oral use can be obtained through combination of glucocorticoid blocker compounds with a solid excipient, optionally grinding a resulting mixture, and processing the mixture of granules, after adding suitable additional compounds, if desired, to obtain tablets or dragee cores. Suitable solid excipients are carbohydrate or protein fillers and include, but are not limited to sugars, including lactose, sucrose, mannitol, or sorbitol; starch from corn, wheat, rice, potato, or other plants; cellulose such as methyl cellulose, hydroxypropylmethyl-cellulose or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

The GR antagonists of this invention can also be administered in the form of suppositories for rectal administration of the drug. These formulations can be prepared by mixing the drug with a suitable non-irritating excipient, which is solid at ordinary temperatures but liquid at the rectal temperatures and will therefore melt in the rectum to release the drug. Such materials are cocoa butter and polyethylene glycols.

The GR antagonists of this invention can also be administered by in intranasal, intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995).

The GR antagonists of the invention can be delivered transdermally, by a topical route, formulated as applicator sticks, solutions, suspensions, emulsions, gels, creams, ointments, pastes, jellies, paints, powders, and aerosols.

The GR antagonists of the invention can also be delivered as microspheres for slow release in the body. For example, microspheres can be administered via intradermal injection of drug (e.g., mifepristone)-containing microspheres, which slowly release subcutaneously (see Rao, J. Biomater Sci. Polym. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The GR antagonist pharmaceutical formulations of the invention can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5, that is combined with buffer prior to use

In another embodiment, the GR antagonist formulations of the invention are useful for parenteral administration, such as intravenous (IV) administration. The formulations for administration will commonly comprise a solution of the GR antagonist (e.g., mifepristone) dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional well-known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of GR antagonist in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the GR antagonist formulations of the invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the GR antagonist into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

B. Dosages

The methods of this invention can be used to ameliorate the symptoms of ALS and/or slow the rate of disease progression. The amount of GR antagonist adequate to accomplish this is defined as a “therapeutically effective dose”. The dosage schedule and amounts effective for this use, i.e., the “dosing regimen,” will depend upon a variety of factors, including the severity of the disease, whether the disease is predominantly restricted to either upper or lower motorneurons, whether the disease is sporadic or familial, the patient's physical status, age and the like. In calculating the dosage regimen for a patient, the mode of administration also is taken into consideration.

The dosage regimen also takes into consideration pharmacokinetics parameters well known in the art, i.e., the GR antagonists' rate of absorption, bioavailability, metabolism, clearance, and the like (see, e.g., Hidalgo-Aragones, J Steroid Biochem. Mol. Biol. 58:611-617, 1996; Groning, Pharmazie 51:337-341, 1996; Fotherby, Contraception 54:59-69, 1996; Johnson, J. Pharm. Sci. 84:1144-1146, 1995; Rohatagi, Pharmazie 50:610-613, 1995; Brophy, Eur. J. Clin. Pharmacol. 24:103-108, 1983; Remington's Pharmaceutical Science, supra). For example, in one study, less than 0.5% of the daily dose of mifepristone was excreted in the urine; the drug bound extensively to circulating albumin (see e.g., Kawai, 1989). The state of the art allows the clinician to determine the dosage regimen for each individual patient, GR antagonist and disease or condition treated. As an illustrative example, the guidelines provided below for mifepristone can be used as guidance to determine the dosage regimen, i.e., dose schedule and dosage levels, of any GR antagonist administered when practicing the methods of the invention.

Single or multiple administrations of GR antagonist formulations can be administered depending on the dosage and frequency as required and tolerated by the patient. The formulations should provide a sufficient quantity of active agent, i.e., mifepristone, to effectively slow the progression of the disease and/or alleviate the symptoms of the disease in a patient diagnosed with ALS. For example, a typical preferred pharmaceutical formulation for oral administration of mifepristone would be about 5 to 15 mg/kg of body weight per patient per day, more preferably between about 8 to about 12 mg/kg of body weight per patient per day, most preferably 10 mg/kg of body weight per patient per day, although dosages of between about 0.5 to about 25 mg/kg of body weight per day may be used in the practice of the invention. Lower dosages can be used, particularly when the drug is administered to an anatomically secluded site, such as the cerebral spinal fluid (CSF) space, into the blood stream, into a body cavity or into the lumen of an organ. Substantially higher dosages can be used in topical administration. Actual methods for preparing parenterally administrable GR antagonist formulations will be known to a person of ordinary skill in the art and are described in more detail in publications such as Remington's Pharmaceutical Science, supra; and Nieman, In Receptor Mediated Antisteroid Action, Agarwal, et al., eds., De Gruyter, N.Y., 1987.

All publications, patents and patent applications cited in this specification are herein incorporated by reference in their entirety as if each individual publication, patent or patent application were specifically and individually indicated to be incorporated by reference.

### EXAMPLES

The following prophetic example is offered to illustrate how to practice the methods of the invention, but not intended to limit the claimed invention.

### Example 1

### Treating a Patient Diagnosed with ALS with Mifepristone

A male patient aged 50 with arm and leg weakness is diagnosed as having ALS. The patients cortisol levels are measured using a blood test and the physician prescribes mifepristone in a dosage of 200 mg daily. The patient's symptoms, cortisol levels, and limb strength is then checked in three weeks. The physician will adjust the dosage of mifepristone if necessary depending on the examination results.

It is understood that the examples and embodiments described herein are for illustrative purposes only and that various modifications or changes in light thereof will be suggested to persons skilled in the art and are to be included within the spirit and purview of this application and scope of the claims

What is claimed is:

1. A method for ameliorating the symptoms and/or slowing the rate of disease progression in a patient diagnosed with amyotrophic lateral sclerosis (ALS), the method comprising administering a therapeutically effective amount of a glucocorticoid receptor specific antagonist (GRA) to a subject in need thereof, with the proviso that the subject not be otherwise in need of treatment with a glucocorticoid receptor antagonist.

2. The method of claim 1, wherein the glucocorticoid receptor antagonist comprises a steroid compound.

3. The method of claim 2, wherein the glucocorticoid receptor antagonist comprises a steroidal skeleton with at least one phenyl-containing moiety in the 11-β position of the steroidal skeleton.

4. The method of claim 3, wherein the phenyl-containing moiety in the 11-β position of the steroidal skeleton is a dimethylaminophenyl moiety.

5. The method of claim 4, wherein the glucocorticoid receptor antagonist is mifepristone.

6. The method of claim 4, wherein the glucocorticoid receptor antagonist is selected from the group consisting of 11β-(4-dimethylaminoethoxyphenyl)-17α-propynyl-17β-hydroxy-4,9-estradien-3-one and 17β-hydroxy-17α-19-(4-methylphenyl)androsta-4,9(11)-dien-3-one.

7. The method of claim 1, wherein the glucocorticoid receptor antagonist is (11β,17β)-11-(1,3-benzodioxol-5-yl)-17-hydroxy-17-(1-propynyl)estra-4,9-dien-3-one.

8. The method of claim 1, wherein the glucocorticoid receptor antagonist is a non-steroidal compound.

9. The method of claim 8, wherein the glucocorticoid receptor antagonist is selected from the group consisting of 1-(o-chloro-α,α-diphenylbenzyl)imidazole; N(triphenylmethyl)imidazole; N-([2-fluoro-9-phenyl]fluorenyl)imidazole; N-([2-pyridyl]diphenylmethyl)imidazole; N-([4,4′,4″]-trichlorotrityl)imidazole; and N((2,6 dichloro-3-methylphenyl)diphenyl)methylimidazole.

10. The method of claim 8, wherein the glucocorticoid receptor antagonist is selected from the group consisting of 6-substituted-1,2-dihydro-N protected-quinoline; octahydrophenanthrenyl carbamate; oxadiazolylalkoxyoctahydrophenanthrene; and octahydrophenanthrene hydrazine.

11. The method of claim 8, wherein the glucocorticoid receptor antagonist is selected from the group consisting of octahydro-2-H-naphthol[1,2,-f]indole-4 carboxamide; cyclopent[f]indazole; and benz[f]indazole.

12. The method of claim 8, wherein the glucocorticoid receptor antagonist is selected from the group consisting of a 6H-dibenzo[b,d]pyran derivative; a substituted aminobenzene derivative; a triphenylmethane derivative; a diphenyl ether derivative; and a modified pyrimidine compound.

13. The method of claim 8, wherein the glucocorticoid receptor antagonist is selected from the group consisting of 1-(2-chlorotrityl)-2-methylimidazole; N-(2-chlorotrityl)-L-prolinol acetate; 1-(2-chlorotrityl)-1,2,4-triazole; and 1-(2-chlorotrityl)-3,5-dimethylpyrazole.

14. The method of claim 8, wherein the glucocortiocoid receptor antagonist is selected from the group consisting of 4α(S)-Benzyl-2(R)-prop-1-ynyl-1,2,3,4,4α,9,10,10α(R)-octahydro-phenanthrene-2,7-diol and 4α(S)-Benzyl-2(R)-chloroethynyl-1,2,3,4,4α,9,10,10α(R)-octahydro-phenanthrene-2,7-diol.

15. The method of claim 1, wherein the glucocorticoid receptor antagonist is an azadecalin or a fused ring azadecalin compound

16. The method of claim 1, wherein the glucocorticoid receptor antagonist is administered in a daily amount of between about 0.5 mg and about 40 mg per kg of body weight per day.

17. The method of claim 1, wherein the glucocorticoid receptor antagonist is administered in a daily amount of between about 5 mg and about 20 mg per kg of body weight per day.

18. The method of claim 1 wherein the administration of the glucocorticoid receptor antagonist is once per day.

19. The method of claim 1 wherein the mode of administration of the glucocorticoid receptor antagonist is selected from the group consisting of: a transdermal application, a nebulized suspension, an aerosol spray, intravenously, intraarterially, intrathecally, intramuscularly and intraperitoneally.

20. The method of claim 1, wherein the mode of administration of the glucocorticoid receptor antagonist is oral.
